(19) United States
(12) Patent Application Publication
(10) Pub. No.: US 2025/0111941 A1
(43) Pub. Date: Apr. 3, 2025

(54) Digital twin of the female pelvic floor

(71) Applicant: Advanced Tactile Imaging, Ewing Township, NJ (US)

(72) Inventor: Vladimir Egorov, Princeton, NJ (US)

(21) Appl. No.: 18/374,721

(22) Filed: Sep. 29, 2023

(51) Int. Cl. G16H 50/20; G06T 7/00; G16H 30/20; G16H 50/50

(52) U.S. Cl. CPC G16H 50/20; G06T 7/0012; G16H 30/20; G16H 50/50; G06T 2207/10088; G06T 2207/10116; G06T 2207/10132; G06T 2207/30004

(57) ABSTRACT

The present invention delineates a comprehensive methodology for the characterization, diagnosis, and treatment decision-making concerning conditions of the female pelvic floor, leveraging a digital twin paradigm. The method entails the acquisition of heterogeneous health data, including but not limited to gynecological, obstetrical, and imaging data, which is subsequently transferred to a remote database. This data repository encompasses various clinical cases and pelvic pathological conditions. A remote machine learning data-processing engine analyzes the collected data to establish a medical diagnosis, predict treatment outcomes, and generate a tailored three-dimensional anatomical model of the patient's pelvic floor. The processed information is returned to the clinician for visualization and serves as the basis for informed treatment decisions. This methodology accommodates multiple imaging modalities, such as Tactile, Ultrasound, Magnetic Resonance, and X-ray Imaging, and considers a wide array of possible treatments and pelvic pathologies. The invention provides the facility for storing all relevant data, diagnosis, treatment probabilities, and the adapted model in a digital twin, facilitating future consultations and treatment planning.

### BACKGROUND

The present invention generally pertains to women's health. More specifically, the invention describes a method for the characterization, diagnosis, and treatment of medical conditions affecting the female pelvic floor.

Pelvic organ prolapse (POP) is an abnormal descent or herniation of the pelvic organs from their normal attachment sites or their normal position in the pelvis. This condition is often associated with concomitant pelvic floor disorders, including urinary and fecal incontinence, pelvic pain, sexual dysfunction, voiding dysfunction, and social isolation. A recent study provided a projection that by the year 2050, 43.8 million women, or nearly 33% of the adult female population in the US, would be affected by at least one troublesome pelvic floor disorder. The lifetime risk of undergoing surgery for POP or urinary incontinence is near 20%.

Urinary incontinence (UI) is a storage symptom and defined as the complaint of any involuntary loss of urine. The most common type of UI is stress urinary incontinence (SUI), defined as the complaint of involuntary leakage on effort or exertion, or on sneezing or coughing. Estimates of the prevalence of this disorder vary depending on the affected population and the definition of UI. Using the inclusive definition of any leakage occurring atleast once, in the past year, the prevalence of UI ranges from 25% to 51%.

The female pelvic floor comprises the pelvic diaphragm muscles (pubococcygeus, puborectalis, and iliococcygeus, together known as the levator ani), the urogenital diaphragm muscles (ischiocavernosus, bulbospongiosus, and transverses perinei superficialis, together known as the perineal muscles); and the urethral and anal sphincter muscles. These muscles interrelate with each other both anatomically and functionally. The normal action of the pelvic floor muscles has been described as a squeeze around the pelvic openings and an inward lift. The pelvic floor disorders result from the neuro-urinary pathology as well as muscle functional impairment due to changes in biomechanical properties of soft tissues associated with age. The anatomy of the pelvic floor is complex and clinical examination alone is often insufficient to diagnose and assess the pathology. That is why a quantitative pelvic floor characterization and diagnosis must include biomechanical measurements. The need exists to improve the characterization of pelvic conditions.

Endometriosis afflicts 6-10% of women of reproductive age, with prevalence of 20-50% in infertile women, and 71-87% of women with chronic pain. The incidence of adenomyosis is reported as 28.9 per 10,000 women. Adenomyosis is detected in 27% of hysterectomy materials in benign gynecological surgery. It leads to abnormal uterine bleeding, pelvic pain, pelvic mass, and infertility. Uterine fibroids are the most common benign tumors of the reproductive system found in 15-65% of women. Like adenomyosis, uterine fibrosis can lead to complaints of vaginal bleeding, pelvic pain, and infertility, depending on their location. Unfortunately, there is often a delay in the diagnosis of such conditions. In patients aged 18-45 years, the average delay is 6.7 years. Early referral, identification of disease and treatment may mitigate pain, prevent disease progression, and thus preserve fertility. Barriers to early diagnosis include the high cost of diagnosis and presentation of confounding symptoms. Thus, a new approach to diagnose such conditions is required to facilitate earlier intervention that could ultimately improve quality of life and preserve fertility.

Preterm delivery is a leading cause of infant mortality and morbidity. An estimated 13.4 million babies were born preterm in 2020. Across countries, the rate of preterm birth ranges from 4% to 16%. It now represents the leading cause of death among children under 5 years of age. Preterm birth is among the most complex and significant challenges in obstetrics. Most preterm births occur spontaneously. Therefore, identifying women at risk for spontaneous preterm birth (sPTB) remains a paramount issue. Premature softening, shortening, and dilation of the cervix, which may be considered early mechanical failure, is associated with sPTB. Clinical findings support the hypothesis that cervical elastography may provide effective biomarkers for predicting preterm delivery. However, in current clinical practice, the progress of cervical ripening is often monitored subjectively through visual inspection and digital examination. The digital cervical score and Bishop score, as predictors of sPTB, demonstrate a low diagnostic accuracy of 61%-68%. Despite numerous risk factors associated with sPTB having been identified in previous decades, the ability to accurately predict when labor will occur remains elusive [18-21]. That is the development of a new approach, which could identify conditions leading to sPTB with a high level of confidence, is required.

The mechanical properties of pelvic tissues undergo significant changes before delivery in pregnant women in order to facilitate the passage of the fetus. However, over 85% of women experience obstetric trauma that impacts one or more components of the pelvic floor, such as the perineal body, levator ani muscles, and the anal sphincter complex. Damage to these structural components can result in pain and serves as the most crucial and potentially modifiable risk factor for subsequent pelvic floor disorders. Preventing or reducing maternal injury presents several challenges, including the inability to (a) evaluate the capacity of key pelvic structures to withstand the strain caused by delivery, (b) predict individual biomechanical risks of maternal injuries prior to delivery, and (c) make informed decisions about obstetrical procedures based on the biomechanical properties of pelvic tissues. To address these challenges, it is essential to (1) characterize the biomechanical status of critical pelvic soft components (such as the perineum and levator ani) under significant stress, (2) measure the anthropometric parameters of the pelvis and fetus in each pregnant woman, and (3) employ validated finite element and risk predictive models. A new approach is required to decrease the likelihood of maternal injury and obstetric trauma at birth.

The current clinical practice for the assessment of pelvic floor disorders is often limited to the evaluation of surface anatomy and manual palpation. Pelvic Organ Prolapse Quantification system (POP-Q) is widely used for describing and staging pelvic support [Bump, R. C., Mattiasson, A., Bo, K., et al. The Standardization of Terminology of Female Pelvic Organ Prolapse and Pelvic Floor Dysfunction. American Journal of Obstetrics and Gynecology 1996; 175:10-17], the Pelvic Floor Distress Inventory (PFDI) and PFDI-20 are recommended by the International Consultation on Incontinence as grade A for assessing pelvic floor dysfunction [de Arruda GT, Dos Santos Henrique T, Virtuoso J F. Pelvic floor distress inventory (PFDI)-systematic review of measurement properties. International Urogynecology Journal 2021; 32 (10): 2657-2669.], the Female Sexual Function Index (FSFI) is used for diagnosing sexual dysfunction in women [Okobi O E. A Systemic Review on the Association Between Infertility and Sexual Dysfunction Among Women Utilizing Female Sexual Function Index as a Measuring Tool. Cureus 2021; 13 (6): e16006.]. In severe or complicated cases, ultrasound, magnetic resonance imaging (MRI), and X-ray imaging may be used for additional evaluation. Bladder and rectum function tests, such as urodynamics, manometry, or defecography might also be employed. Vaginal tactile imaging and tactile ultrasound image fusion provide a set of biomechanical parameters to quantify vaginal tissue elasticity, pelvic support strength and function [Egorov V. Biomechanical Mapping of the Female Pelvic Floor. 1st Ed, Elsevier Academic Press, 2023:1-394. ISBN: 9780323851619]. The Biomechanical Integrity score (BI-score) of the female pelvic floor was introduced [Egorov V, van Raalte H, Takacs P, Shobeiri S A, Lucente V, Hoyte L. Biomechanical integrity score of the female pelvic floor. International Urogynecology Journal 2022; 33:1617-1631.].

The true etiology of POP and variations observed among individuals are not entirely understood. The pelvic disorders are thought to share common pathogeneses, tissue elasticity changes, weakening of the connective support tissues, and pelvic muscle dysfunction. Logically to propose that a biomechanical assessment and characterization of the female pelvic floor could bring important information in clinical practice. However, ultrasound and MRI elastography, as well as functional imaging of the pelvic floor, did not get appropriate acceptance in Urogynecology. There is a significant gap in biomechanical and functional research of the female pelvic floor.

Treatment options for POP and SUI include surgery and pelvic muscle training. An invasive surgical approach is considered the ultimate treatment for both POP and SUI. It was found that surgical treatments of recurrent SUI are associated with high failure rates. Physical training and medications are often not effective. There is a need for the objective and quantitative pre- and post-surgery assessment of pelvic floor conditions to improve evidence-based management in urogynecological surgeries.

Vaginal Tactile Imager (VTI) was developed to provide a biomechanical mapping of the pelvic floor with a vaginal probe. A set of new clinical markers/parameters has been proposed for the biomechanical characterization of the pelvic floor conditions [Sarvazyan N, Francy B, Egorov V. Vaginal Tactile Imaging: A Review. Pelviperineology 2023; 42 (1): 28-42]. This set included 52 parameters automatically calculated in results of the completion of eight examination procedures (tests). To make the biomechanical mapping in urogynecology more accessible and useful, further work is required on developing an automatic framework to deliver pelvic floor diagnosis and prediction of treatment outcome.

Vaginal Tactile Ultrasound Imager (TIUSv) was developed to provide a comprehensive anatomical and biomechanical mapping of the pelvic floor with a vaginal probe. A set of new clinical markers/parameters has been proposed for characterization of the pelvic floor conditions [Egorov V, van Raalte H, Shobeiri S A. Tactile and ultrasound image fusion for functional assessment of the female pelvic floor. Open Journal of Obstetrics and Gynecology 2021; 11:674-688]. This set included 68 parameters automatically calculated in results of the completion of eight examination procedures (tests). To make this approach more accessible and useful, further work is required on developing an automatic framework to deliver pelvic floor diagnosis and prediction of treatment outcome.

Cervix Monitor (CM) was designed and validated in bench testing and a clinical study. The CM includes a detachable data acquisition probe, a signal generating/acquisition unit, and a touchscreen computer. The CM probe comprises the tactile array with four sensors and ultrasound transducer. The clinical study demonstrated that cervical stress-strain data allowed the computation of cervical elasticity and length. This approach has the potential to provide cervical markers to predict spontaneous preterm delivery [Egorov V, Rosen T, van Raalte H, Kurtenoks V. Cervical characterization with tactile-ultrasound probe. Open Journal of Obstetrics and Gynecology 2020; 10:85-99]. Further work is required on developing a framework with a predictive model to deliver reliable detection of pelvic conditions leading to spontaneous preterm birth.

Antepartum Tactile Imager (ATI) was designed and validated in bench testing and a clinical study. The ATI is based on the acquisition of stress-strain data through a tactile and motion tracking system, and enables the assessment of biomechanical parameters for the critical structures, as well as the anatomical characterization of the birth canal at its critical cross-section [Rusavy Z, Kalis V, Aglyamov S, Egorov V. Feasibility and safety of antepartum tactile imaging. International Urogynecology Journal 2021; 32:1785-1791]. A new framework is required to establish correlations between biomechanical/anatomical data on critical pelvic structures before vaginal delivery and birth injury outcome statistics. Additionally, there is a need to develop a risk model of maternal birth injury for various pre-delivery conditions, including fetal weight/size, and applied obstetrical procedures and delivery mode.

While the terminology has changed over the years, the concept of creating a digital and physical twin as an entity duplication has been practiced since the 1960s. The National Aeronautics and Space Administration (NASA) has utilized twinning ideas to match the systems in space. An example of this is when NASA developed a digital twin to assess and simulate conditions on board Apollo 13. In 2002, Dr. Grieves proposed the creation of physical products, virtual products, and a data interface between them in the context of product life cycle management (PLM) as a conceptual ideal [Grieves M. Product Lifecycle Management: The new paradigm for enterprises. International Journal of Product Development 2005; 2 (1/2): 71-84]. In 2011, the U.S. Air Force Research Laboratory applied the digital twin concept as ‘a reengineering of structural life prediction and management’ to an airplane frame [Tuegel E J, Ingraffea A R, Eason T G, Spottswood S M. Reengineering Aircraft Structural Life Prediction Using a Digital Twin. Hindawi Publishing Corporation. International Journal of Aerospace Engineering 2011; Article ID 154798; 1-14]. Since then, the digital twin concept has extended to sectors such as transportation, industrial production, intelligent education, and others. Eleven definitions of a digital twin were proposed from 2012 to 2022. One of them is: “A digital twin is a real-time mapping of physical, virtual, and interaction data in the entire life cycle of a physical entity” [Tao F, Cheng J F, Qi Q L, Zhang M, Zhang H, Sui F Y. Digital twin-driven product design, manufacturing and service with big data. Int J Adv Manuf Technol 2018; 94 (9): 3563-76].

In the last two years, research publications have explored various applications of digital twin in medicine. These include reporting the potential of a medical digital twin in diabetes management [Chu Y, Li S, Tang J, Wu H. The potential of the Medical Digital Twin in diabetes management: a review. Front Med (Lausanne) 2023; 10:1178912]; detailing recent advances in home monitoring of patients with asthma using digital twin systems [Drummond D, Roukema J, Pijnenburg M. Home monitoring in asthma: towards digital twins. Curr Opin Pulm Med 2023; 29 (4): 270-76]; outlining the use of digital twins for predictive, preventive, personalized, and participatory treatment of immune-mediated diseases [Benson M. Digital Twins for predictive, preventive personalized, and participatory treatment of immune-mediated diseases. Arterioscler Thromb Vasc Biol. 2023; 43 (3): 410-16]; presenting the original concept of controlling a rehabilitation exoskeleton via its digital twin in virtual reality to be used for manual remote kinesiotherapy combined with safety systems predicting potentially harmful situations [Falkowski P, Osiak T, Wilk J, Prokopiuk N, Leczkowski B, Pilat Z, Rzymkowski C. Study on the applicability of digital twins for home remote motor rehabilitation. Sensors (Basel) 2023; 23 (2): 911]; describing digital twin systems to be developed for real-time monitoring and management of children with chronic diseases [Drummond D, Coulet A. Technical, ethical, legal, and societal challenges with digital twin systems for the management of chronic diseases in children and young people. J Med Internet Res. 2022; 24 (10): e39698]; defining digital twin requirements for a liquid-filled incubator to mimic the environment of the natural womb for premature infants [van Willigen B G, van der Hout-van der Jagt M B, Huberts W, van de Vosse F N. A review study of fetal circulatory models to develop a digital twin of a fetus in a perinatal life support system. Front Pediatr. 2022; 10:915846]; highlighting potential benefits of incorporating digital twins to support better dementia care [Wickramasinghe N, Ulapane N, Andargoli A, Ossai C, Shuakat N, Nguyen T, Zelcer J. Digital twins to enable better precision and personalized dementia care. JAMIA Open 2022; 5 (3): ooac072]; presenting opportunities and challenges of applying digital twins in clinical oncology, particularly focusing on integrating medical imaging with mechanism-based, tissue-scale mathematical modeling [Wu C, Lorenzo G, Hormuth D A 2nd, Lima EABF, Slavkova K P, DiCarlo J C, Virostko J, Phillips C M, Patt D, Chung C, Yankeelov T E. Integrating mechanism-based modeling with biomedical imaging to build practical digital twins for clinical oncology. Biophys Rev (Melville). 2022; 3 (2): 021304]; and describing the development of digital twins derived from three-dimensional scanning data to measure soft tissue deformation in head and neck surgery using an artificial intelligence approach [Männle D, Pohlmann J, Monji-Azad S, Hesser J, Rotter N, Affolter A, Lammert A, Kramer B, Ludwig S, Huber L, Scherl C. Artificial intelligence directed development of a digital twin to measure soft tissue shift during head and neck surgery. PLOS One 2023; 18 (8): e0287081]. The application of the digital twin to the cardiovascular system includes the establishment of digital twin heart models and the precise treatment of cardiovascular disease [Sun T, He X, Li Z. Digital twin in healthcare: Recent updates and challenges. Digit Health 2023; 9:20552076221149651]. The use of digital twins in healthcare is an emerging topic. This can be seen in the limited literature supporting this technology.

### SUMMARY

The objective of the present invention is to overcome the drawbacks of the prior art and to provide novel methods for characterization of the female pelvic floor conditions.

Another objective of the present invention is to overcome the drawbacks of the prior art and to provide novel methods for diagnosis of medical conditions in the female pelvic floor.

Another objective of the present invention is to overcome the drawbacks of the prior art and to provide novel methods for prediction of treatment outcomes of the female pelvic floor.

Another objective of the invention is to provide novel methods for characterization of pelvic organ prolapse development in women.

Another objective of the invention is to provide novel methods for characterization of stress urinary incontinence in women.

Another objective of the invention is to provide novel methods for characterization of vaginal tissue atrophy in women.

Another objective of the invention is to provide novel methods for characterization of chronic pelvic pain in women.

Another objective of the invention is to provide novel methods for the detection and characterization of endometriosis in the female pelvic floor.

Another objective of the invention is to provide novel methods for the detection and characterization of adenomyosis in the female pelvic floor.

Another objective of the invention is to provide novel methods for the detection and characterization of uterine fibroids in the female pelvic floor.

Another objective of the invention is to provide novel methods for the detection and characterization of cervical cancer in the female pelvic floor.

Another objective of the invention is to provide novel methods for the detection and characterization of cervical conditions in pregnant women leading to spontaneous preterm birth.

Another objective of the invention is to provide novel methods for characterization of a birth canal in pregnant women to predict maternal trauma for a set of obstetrical procedures during vaginal delivery and delivery mode.

According to the present invention, novel methods for characterization of female pelvic with a digital twin may include the following the steps:
(a) acquisitioning heterogeneous health data related to the female pelvic floor,
(b) transferring said health data to a remote,
(c) analyzing said health data by a remote machine learning data-processing engine to establish a medical diagnosis, treatment outcomes and prepare a three-dimensional model adapted for the female pelvic floor conditions,
(d) returning said medical diagnosis, the probabilities of treatment success and the adapted three-dimensional model,
(e) visualizing said diagnosis, the probabilities of treatment success and the three-dimensional model to clinician and patient,
(f) making a decision about pelvic floor treatment of the patient based on the diagnosis and the probabilities of treatment success, and
(g) storing the health data, said diagnosis, the probabilities of treatment success and the adapted three-dimensional model in the remote database as a dated page in said digital twin.

### BRIEF DESCRIPTION OF DRAWINGS

The subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through the use of the accompanying drawings, in which:

FIG. 1 is a principal diagram of an embodiment of the invention which presents basic components of a digital twin for the female pelvic floor;

FIG. 2 is an illustration of sources to acquire heterogeneous pelvic health data.

FIG. 3 shows a flow chart illustrating a method for characterization of the female pelvic floor conditions with a digital twin.

### DETAILED DESCRIPTION OF THE INVENTION

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, components and/or circuits have not been described in detail to avoid unnecessarily obscuring claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

In this invention, a digital twin of the female pelvic floor is defined as a healthcare framework which integrates:
(1) a patient examination with available means to acquire heterogeneous pelvic health data,
(2) a growing variety of data for women and their pelvic diseased conditions,
(3) a machine learning data-processing engine to provide clinical diagnosis and predict pelvic treatment outcomes,
(4) a three-dimensional (3D) anatomical model of the female pelvic floor,
(5) a tool for visualization of a clinical diagnosis in the 3D anatomical model,
(6) a tool for visualization of pelvic treatment outcomes, and
(7) a dated digital twin page with the acquired pelvic data, the diagnosis, the adapted 3D anatomical model and the predicted treatment outcomes.

The female pelvic floor is a complex structure consisting of muscles, ligaments, and connective tissue, providing support for various organs such as the bladder, uterus, and rectum. Disorders of the pelvic floor, such as incontinence, organ prolapse, tissue atrophy, chronic pelvic pain, overactive bladder, endometriosis, adenomyosis, uterine fibroids, cervical cancer, and cervical mechanical deficiency for pregnant women are prevalent and distressing conditions affecting women of all ages. Traditional diagnostic methods, like physical examination or imaging, may not fully capture the nuanced characteristics of individual patients' conditions. The use of the digital twin of the female pelvic floor can revolutionize the diagnosis, treatment, and long-term management of these conditions.

The digital twin of the female pelvic floor involves integrating all available objective and subjective data for a specific patient. Objective data may include imaging findings from tactile, ultrasound, magnetic resonance imaging (MRI), or computer tomography (CT) scans, along with data from physiological tests such as urodynamic studies, dynamic tactile imaging and biomechanical mapping. Subjective data might include patient-reported symptoms, personal medical history, lifestyle factors, and quality of life measures.

Tactile imaging and biomechanical mapping are crucial aspects of developing a comprehensive digital twin of the female pelvic floor. These sophisticated technologies add valuable layers of information that increase the model's anatomical precision and functional details. Tactile imaging captures the response of soft biological tissues to applied pressure or touch. This advanced technique can provide a detailed topographical and tomographical mapping of the tissue elasticity, crucial in understanding the pelvic floor's biomechanical properties. In the context of the female pelvic floor, tactile imaging can help identify areas of abnormal rigidity or laxity which could be contributing to conditions like prolapse or incontinence.

Biomechanical mapping, on the other hand, involves the quantitative analysis of the forces, stresses, and strains operating in the pelvic floor structures. This may involve complex computer simulations based on the individual's anatomy and tissue properties. In addition to the anatomical modeling, biomechanical mapping contributes significantly to functional insights, highlighting how the pelvic floor muscles and connective tissues work together during various activities, such as standing, lifting, or coughing.

Incorporating tactile imaging and biomechanical mapping into the digital twin of the female pelvic floor adds an exceptional level of detail and personalization. Not only do these modalities improve the structural accuracy of the model, but they also offer dynamic, functional insights. By simulating how the pelvic floor responds to different physical stresses, they allow for the prediction of potential failure points and vulnerable areas that may be at risk for injury or dysfunction.

From a clinical perspective, these technologies enhance the benefits previously outlined. The added precision can further improve diagnostic accuracy and personalize treatment planning, while the dynamic, functional insights can enhance patient education and long-term monitoring. For instance, using biomechanical simulations, clinicians could demonstrate to a patient how specific physical activities or behaviors might be straining their pelvic floor, guiding lifestyle modifications. Similarly, these technologies can support the design of personalized physiotherapy programs, adjusting exercises to the patient's specific biomechanical profile. Furthermore, by tracking changes in tactile imaging and biomechanical maps over time, clinicians can monitor how the pelvic floor is responding to treatment or how it is changing with age, pregnancy, or other life events. This can prompt timely modifications to the treatment plan and prevent the development or progression of pelvic floor disorders.

Once these data are integrated, sophisticated machine learning algorithms can generate a 3D anatomical and functional image of the patient's pelvic floor. This model represents the “twin” of the actual pelvic floor, providing an unprecedented level of detail and offering a comprehensive picture of the patient's condition. The clinical benefits of this approach are significant. Firstly, it enhances diagnostic accuracy. The digital twin provides a more holistic and nuanced understanding of the patient's condition, reducing diagnostic uncertainty, and leading to more targeted treatment strategies. Secondly, it improves treatment planning. The digital twin can be used to predict the impact of different treatments on the pelvic floor. This predictive modeling can aid clinicians in tailoring treatments to individual patient needs and anticipate potential complications or outcomes. Thirdly, it facilitates patient education and engagement. By visualizing their condition in a 3D model, patients can gain a better understanding of their condition, increasing their involvement in the treatment decision-making process and enhancing their adherence to the prescribed therapy. Finally, the digital twin can be continuously updated with new data over time, providing a dynamic, long-term view of the patient's condition. This ongoing monitoring can help track the progression of the disease and the effectiveness of treatments, supporting timely adjustments to the care plan as necessary.

The digital twin of the female pelvic floor can be a powerful tool in personalized medicine. By integrating a range of data into a comprehensive 3D model, it enhances diagnostic accuracy, facilitates personalized treatment planning, promotes patient engagement, and supports long-term patient monitoring, significantly improving the care and outcomes for women with pelvic floor disorders.

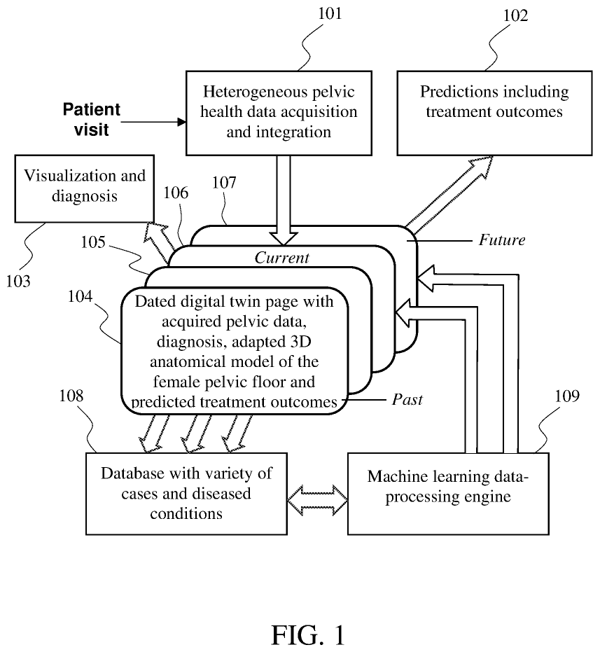

FIG. 1 illustrates a schematic diagram of an embodiment of the invention, which delineates the essential components of a digital twin for the female pelvic floor. The digital twin encompasses heterogeneous pelvic health data (101) garnered during the examination of a patient by available means. This heterogeneous pelvic health data is integrated into a current digital twin page (105) and subsequently transferred to a remote database (108) containing a diverse array of clinical cases and pathological conditions related to pelvic health. A machine-learning data-processing engine (109) furnishes clinical diagnoses, facilitates the adaptation of a three-dimensional anatomical model of the female pelvic floor tailored to the examined patient, and predicts treatment outcomes across various therapeutic modalities and treatment. The model adaptation incorporates anatomical changes in the 3D model pertinent to the clinical diagnosis. Subsequently, the clinical diagnosis, the adapted 3D anatomical model, and the prediction of pelvic treatment outcomes are channeled to the current digital twin page (105). Thereafter, both the clinical diagnosis and the adapted 3D anatomical model are relayed to a visualization tool (103), while the prediction of pelvic treatment outcomes is transferred to a specialized tool for visualizing said treatment outcomes. Clinicians and patients may interact, either physically or virtually, with these visualization tools (102 and 103) to deliberate on these findings and concur on a treatment pathway. Future iterations of digital twin pages, facilitated by the machine-learning data-processing engine (109), may include a short-term (1-2 years) page (106) and a mid-term (3-5 years) page, both projecting pelvic treatment outcomes for the selected treatment route.

Further, the digital twin may include a finite element model (FEM) for tissue and pelvic organ deformation by an applied probe for data acquisition. using a commercial software package, COMSOL Multiphysics (COMSOL, Stockholm, Sweden). The nonlinear elastic properties of the pelvic floor may be described with a hyper-elastic Neo-Hookean model with Poisson's ratio close to 0.5. The birth canal, including the perineum and the levator ani muscle, may be modeled as an incompressible, hyper-elastic layer. The Young's modulus of the soft tissues may be calculated using the applied probe anatomy and FEM results.

The 3D anatomical model of the female pelvic floor may be built with the use of Blender software (Blender Foundation, Amsterdam, Netherland), which enables digital sculpting tools with flexibility required in future anatomical adaptations to recorded tactile and ultrasound images. By offering the sculpting and the polygonal modeling toolsets side by side, Blender greatly simplifies the transition between conceptual research and final design of dynamic models with interactive modifications, fast object, shader, light changes, tiled and progressive rendering.

The 3D anatomical model of the female pelvic floor may encompass the following clinically important aspects. Within the pelvis, two distinct compartments (anterior and posterior) are defined by the vaginal walls, which anchor to the pelvic sidewalls. The anterior compartment contains the bladder and urethra, with the pubic bones and pelvic sidewalls forming inflexible anterior and lateral boundaries. Thus, the flexible components of this compartment include the anterior vaginal wall, urethra, bladder, and ligaments. In contrast, the posterior compartment accommodates the rectum and anus. Unlike the anterior compartment, only the sacrum is inflexible in the posterior compartment, with deformable structures such as the levator ani muscles, the perineal body, and the posterior vaginal wall. Closure of the external openings to the bladder and rectum is managed by the urethral sphincter and anal sphincter, respectively. The levator ani muscles function to seal the entire pelvic cavity, including the vagina. When at rest, these muscles keep the pelvic organs closed and sustain the weight imposed by the abdominal and pelvic organs above. The pelvic muscles have the ability to be voluntarily squeezed or to involuntarily react to stimuli like coughing. They actively resist forces created during events like the Valsalva maneuver and straining. The support system of the pelvic floor is intricate and interdependent, composed of muscles and connective tissues. This system embodies the pelvic organs, their structural connections to the pelvis, and a neuromuscular mechanism that dynamically responds to changes in force. Such forces can result from the weight of the pelvic organs themselves or from pressure differentials that might arise between abdominal and atmospheric pressures.

Despite significant progress in the comprehension of female pelvic floor anatomy, encompassing functional facets, discrepancies persist regarding anatomy, neural innervations, and the functional assessment of pelvic floor muscles. This lack of consensus represents a substantial impediment to the evolution of understanding and treatment of pelvic floor disorders. In this invention, the interdependence between pelvic organ support systems, correlating ligament-fascia lesions with clinical manifestations are used to facilitate an integrated representation of the pelvic floor structures. A crucial dimension of the 3D model is the linkage of the mechanical and functional properties of each anatomical structure with the biomechanical parameters ascertained in patient examination using the probes delineated in the above. Specifically, the 3D anatomical digital model may comprise muscles, ligaments, fascia, and the visceral system. The pelvic floor muscles will be categorized into three distinct layers. The first layer, or the most external, is known as the urogenital triangle, and is composed of the bulbocavernosus, ischiocavernosus, and external anal sphincter muscles. The middle layer is referred to as the urogenital diaphragm or perineal membrane, and consists of the superficial transverse perineal, deep transverse perineal, and sphincter urethrae muscles. The third layer, or the deep layer, is the pelvic diaphragm, comprising the levator ani and coccygeus muscles. The levator ani muscle itself is further may be segmented into three constituent muscles: pubococcygeus, ileococcygeus, and puborectalis.

The 3D digital model further may include four compartments: anterior, middle, posterior, and peritoneum. The anterior compartment will encompass the bladder and urethra; the middle compartment will include the vagina and uterus; the posterior compartment will house the anus and rectum; and the peritoneum will consist of the endopelvic fascia and perineal membrane. Scientific evidence has suggested the potential involvement of the surrounding fasciae and ligaments in the pathophysiology of pelvic floor disorders. For instance, the pelvic floor fasciae are thought to contribute to urethral and pelvic organ support, thereby preventing incontinence and prolapse. The 3D model may include the pubourethral and pubovesical ligaments, tendinous arch of pelvic fascia, tendinous arch of levator ani, pubocervical fascia, rectovaginal fascia, tendinous arch of rectovaginal fascia, rectovaginal ligament, rectosacral fascia, and the inferior fascia of the pelvic diaphragm, along with the cardinal and uterosacral ligaments. All pelvic anatomical components may have deformable connections.

The fundamental aspect of the digital twin is creating a realistic digital representation of a patient's anatomy for utilization in device measurement, clinical information gathering, data analysis, visualization, and tracking changes. The process of building this digital analogue commences with the selection of an appropriate initial digital base model. Subsequently, transformations such as scaling, skewing, and inflation/deflation are applied to the digital anatomy and its constituent parts. In this invention, parameters for the digital twin are derived from an anatomy description form completed by the physician, coupled with acquired measurement data from the applied device. A set of anatomical parameters serves as a generalized representation of the 3D model for the digital twin. A digital twin software facilitates the understanding of a patient's problems. It is employed to visualize specific issues during discussions with the patient, visually track changes, and monitor treatment progress over time. Once set up, it may be used interactively during examination procedures. The results of patient examination with the appropriate device, or any other data field that can be represented on the surface of the digital anatomy, may be displayed as textures on the model surface in the form of colored patterns. Multiple fields can be visualized using special techniques for color blending or mixing colors and patterns. Possessing a digital model of the anatomy affords a much better understanding of the underlying conditions because physician is not constrained by physical 3D space, and spatial transformations may be applied to the digital model to reveal hidden surfaces. Possible options include the simulation of endoscopy or the transformation of a 3D surface into a 2D image. Interpolation and extrapolation may be used to build intermediate and projected model modification. The following functionalities may be used: (1) parsing geometry files built in OpenGL; (2) creating signal mapping as signal fields to 3D anatomy objects; (3) applying signal fields to the geometry as colored textures, with potential blending with the distinctive colors of anatomy parts, and/or as scaled anatomy distortion; (4) 3D view modifications to allow for general modification of the 3D view with relative scaling of anatomy parts by applying generic transformations of coordinates, scaling, and rotation, thereby providing access to hidden parts of the anatomy, which may include interactive modifications by the user, such as cross-sections and selections of muscles and ligaments; (5) working modes including both post-acquisition, where accumulated and processed data are visualized, and interactive modes during data acquisition; (6) tools and markers to pinpoint locations on the surface, display information related to specific points, show details related to multiple points (such as distance, area, pressure, and pressure gradient), and enable dual visualization for comparison; (7) analysis/measurement to enable reverse conversion to optimize geometry transformation based on acquired data, and provide estimated parameters for this geometry transformation.

Further, the 3D anatomical model may consist of a combination of scaled digital mandatory anatomical components, with the optional addition of local transformations related to known issues. These scaling parameters and transformation options form the foundation for the 3D anatomical model definition. The data-processing engine may utilize these parameters in its learning neural network, and changes to the parameters over time may be monitored to create a historical record of patient treatment in the dated digital twin page. A 3D model of the female pelvic floor may be constructed for any specific point in time, providing a representation more familiar to the physician. The extrapolated values from such models may be employed for comparing the estimated current state with the results of the last examination. This comparison enables corrections to initial diagnostic assumptions and parameters of the digital twin representation, thereby enhancing the engine training. An artificial machine learning network may be trained on available clinical data for pelvic treatment outcomes, with the goal of improving a predictive treatment model.

In this invention, software procedures and algorithms translate pelvic examination data (measurements) into the dated digital twin page. The following features extraction may be used in medical image classification: texture characteristics such as correlation, contrast, homogeneity, cluster-shade, and dissimilarity, and shape and intensity characteristics, and motion descriptors. Further, unsupervised machine learning techniques may be used to identify patterns and trends within unlabeled patient examination data. Additionally, focus on clustering data based on similarities or differences, discerning underlying patterns within datasets, and detecting anomalies and outliers may be employed. A variety of feature extraction models, as well as image classification and image segmentation models might be used for the digital twin. Principal component analysis, a linear dimension-reduction technique, may be employed to perform an orthogonal transformation that converts a set of correlated features into a set of uncorrelated variables.

A supervised machine learning model, referred to as a classifier, may be used in the digital twin with multiple objectives: to differentiate between diseased and normal conditions of the pelvic floor, to prognosticate potential disease development based on current conditions, and to generate a medical diagnosis by determining the presence of disease based on examination data. The machine learning data-processing engine may use algorithms that efficiently solve classification problems of various types such as a K-nearest neighbor method, artificial neural networks decision trees and random forests, as well as genetic algorithms. Also, a reinforcement learning model for treatment outcome predictions may be used as the computational approach of learning from action by interacting with the digital twin through trial and error and receiving rewards (negative or positive) as feedback. Additionally, finite element modeling may be used further refined and incorporated into the TIUSv, ATI, and CM analytical software modules.

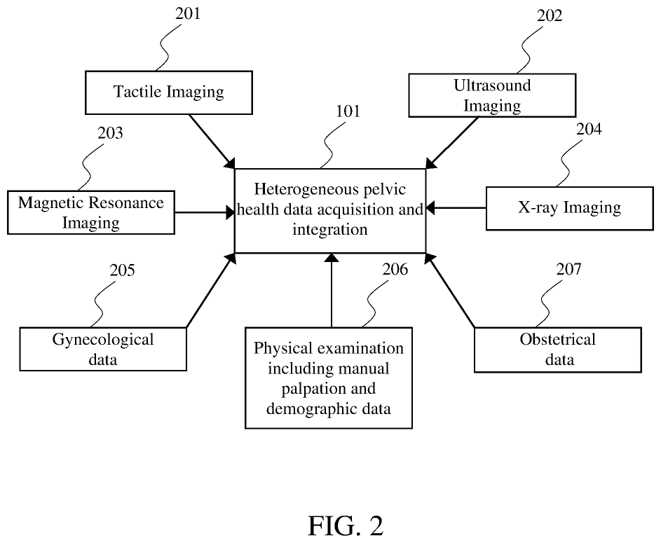

FIG. 2 is an illustration of sources to acquire heterogeneous pelvic health data. Among these sources or means are vaginal tactile imaging (201), ultrasound imaging (202), magnetic resonance imaging (203), X-ray imaging or computer tomography (204), clinical gynecological data (205), obstetrical historic data (207), and physical examination data including manual palpation and demographic data (206) for the examined patient. Examples of vaginal tactile imaging devices to be used for acquisition of the pelvic health data are Vaginal Tactile Imager (VTI), Vaginal Tactile Ultrasound Imager (TIUSv), Antepartum Tactile Imager (ATI), Cervix Monitor (CM) and Stress Strain Imager (SSi). All these devices serve as measuring tools for the female pelvic floor. The VTI allows for acquisition of 52 biomechanical parameters for the female pelvic floor sub-divided into five components to characterize: (1) tissue elasticity, (2) pelvic support, (3) pelvic muscles contractive strength, (4) involuntary muscle relaxation (innervation), and (5) muscle mobility. These components contribute into the Biomechanical Integrity score (BI-score) of the female pelvic floor. The TIUSv allows for acquisition of 68 biomechanical parameters including 16 ultrasound parameters and tactile ultrasound image fusion to characterize tissue elasticity pelvic support and functions. The ATI system enables the acquisition of 3D pressure (stress) feedback patterns and motion tracking (strain) data. This allows for the assessment of biomechanical parameters in critical pelvic structures, such as the levator ani and perineum, as well as the anatomical characterization of the birth canal at its essential cross-sections, prior to vaginal delivery. The CM allows for acquisition of stress-strain and cervical length data to be used in a predictive model for spontaneous preterm delivery. The SSi allows for acquisition of stress-strain data at significant controllable pelvic tissues deformation (up to 30 mm) for detection and characterization of endometriosis, adenomyosis, and uterine fibroids. This data combination allows biomechanical mapping of the soft tissues in absolute scale. Tactile and ultrasound image fusion has a special fundamental importance because of the complementary nature of these technologies; tactile images provide stress data and ultrasound images provide strain data for the same region during tissue deformation. This allows elasticity assessment as a stress to strain ratio-physical parameter of tissue with high sensitivity to the pathology development.

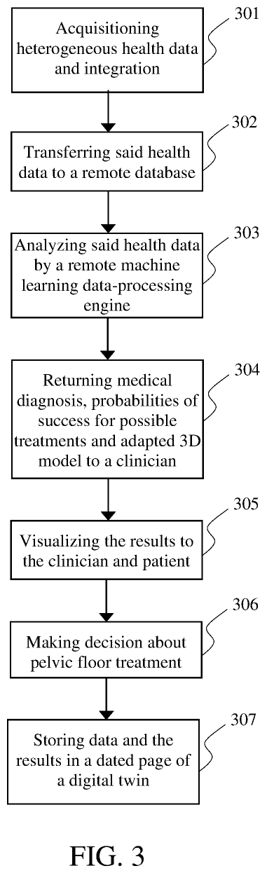

FIG. 3 shows a flow chart illustrating a method for characterization of the female pelvic floor conditions with a digital twin according to the present invention, the method comprising the steps of: (a) acquisitioning heterogeneous health data (301) related to the female pelvic floor of a patient by a clinician and said health data integration in a predetermined structured format; (b) transferring said health data (302) to a remote database with a variety of clinical cases and pelvic diseased conditions; (c) analyzing said health data (303) by a remote machine learning data-processing engine to establish a medical diagnosis, to estimate probabilities of success for possible treatments of the said female pelvic floor conditions and prepare a three-dimensional model adapted for said female pelvic floor conditions; (d) returning (304) said medical diagnosis, the probabilities of success for said possible treatments and the adapted three-dimensional model from the data-processing engine to the clinician; (e) visualizing (305) said diagnosis, the probabilities of success for said possible treatments and the adapted three-dimensional model to the clinician and the patient; (f) making a decision (306) about pelvic floor treatment of the patient based on said diagnosis and the probabilities of success for said possible treatments; and (g) storing (307) said heterogeneous health data, said diagnosis, the probabilities of success for possible treatments and the adapted three-dimensional model in said remote database as a dated page in said digital twin.

The step (a) further may comprise acquisition of said health data for the female pelvic floor with an any combination of the following imaging modalities: Tactile imaging, Ultrasound imaging, Magnetic Resonance Imaging and X-ray Imaging, wherein said imaging modalities provide health data for a stress-strain load and elastography on the female pelvic floor tissues and support structures. Further, said imaging modalities may provide health data for an any combination of functional tests on the female pelvic floor such as voluntary pelvic muscle contraction, reflex pelvic muscle contraction at cough, Valsalva maneuver, and involuntary pelvic muscle relaxation. The step (a) further may comprise acquisition of said health data for the female pelvic floor as a combination of gynecological data, obstetrical historical data, demographic data and physical examination data including manual palpation for the patient. The step (b) further may comprise an any combination of pelvic organ prolapse, stress urinary incontinence, tissue atrophy, chronic pelvic pain, overactive bladder, endometriosis, adenomyosis, uterine fibroids, cervical cancer, and cervical mechanical deficiency for pregnant women as said pelvic diseased conditions. The step (c) further may comprise an any combination of the following said possible treatments: pelvic floor muscle exercises, physical therapy, lifestyle modifications, pessaries, pharmacotherapy, sacral colpopexy, sacrospinous ligament suspension, uterosacral ligament suspension, iliococcygeal suspension, anterior colporrhaphy, posterior colporrhaphy, enterocele repair, perineorrhaphy, Burch procedure, total hysterectomy, supracervical hysterectomy, and mid-urethral sling procedure. The steps (d) and (e) further may comprise returning and visualizing said medical diagnosis, the probabilities of success for said possible treatments and the adapted three-dimensional model to a medical device which was used in acquisition of said health data for the female pelvic floor. The step (f) further may comprise making a decision about pelvic floor treatment of the patient based on maximum probability of success for at least one of said possible treatments. The step (g) further may comprise a set of said dated pages in said digital twin mapping of physical and virtual data during a life cycle of the female pelvic floor.

It is contemplated that any embodiment discussed in this specification can be implemented with respect to any method of the invention, and vice versa. It will be also understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All publications mentioned in the specification are indicative of the level of skill of those skilled in the art to which this invention pertains. All publications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12, 15, 20 or 25%.

All of the devices and/or methods disclosed and claimed herein can be made and executed without undue experimentation in light of the present disclosure. While the devices and methods of this invention have been described in terms of preferred embodiments, it will be apparent to those of skill in the art that variations may be applied to the devices and/or methods and in the steps or in the sequence of steps of the method described herein without departing from the concept, spirit, and scope of the invention. All such similar substitutes and modifications apparent to those skilled in the art are deemed to be within the spirit, scope, and concept of the invention as defined by the appended claims.

What is claimed is:

1. A method for characterization of female pelvic floor with a digital twin, said method comprising the steps of:
(a) acquisitioning heterogeneous health data related to the female pelvic floor of a patient by a clinician and said health data integration in a predetermined structured format;
(b) transferring said health data to a remote database with a variety of clinical cases and pelvic diseased conditions;
(c) analyzing said health data by a remote machine learning data-processing engine to establish a medical diagnosis, to estimate probabilities of success for possible treatments of the said female pelvic floor conditions and prepare a three-dimensional model adapted for said female pelvic floor conditions;
(d) returning said medical diagnosis, the probabilities of success for said possible treatments and the adapted three-dimensional model from the data-processing engine to the clinician;
(e) visualizing said diagnosis, the probabilities of success for said possible treatments and the adapted three-dimensional model to the clinician and the patient;
(f) making a decision about pelvic floor treatment of the patient based on said diagnosis and the probabilities of success for said possible treatments; and
(g) storing said heterogeneous health data, said diagnosis, the probabilities of success for possible treatments and the adapted three-dimensional model in said remote database as a dated page in said digital twin.

2. The method as in claim 1, wherein said step (a) further comprising acquisition of said health data for the female pelvic floor with an any combination of the following imaging modalities: Tactile imaging, Ultrasound imaging, Magnetic Resonance Imaging and X-ray Imaging.

3. The method as in claim 2, wherein said imaging modalities provide health data for a stress-strain load and elastography on the female pelvic floor tissues and support structures.

4. The method as in claim 2, wherein said imaging modalities provide health data for an any combination of functional tests on the female pelvic floor such as voluntary pelvic muscle contraction, reflex pelvic muscle contraction at cough, Valsalva maneuver, and involuntary pelvic muscle relaxation.

5. The method as in claim 1, wherein said step (a) further comprising acquisition of said health data for the female pelvic floor as a combination of gynecological data, obstetrical historical data, demographic data and physical examination data including manual palpation for the patient.

6. The method as in claim 1, wherein said step (b) further comprising an any combination of pelvic organ prolapse, stress urinary incontinence, tissue atrophy, chronic pelvic pain, overactive bladder, endometriosis, adenomyosis, uterine fibroids, cervical cancer, and cervical mechanical deficiency for pregnant women as said pelvic diseased conditions.

7. The method as in claim 1, wherein said step (c) further comprising an any combination of the following said possible treatments: pelvic floor muscle exercises, physical therapy, lifestyle modifications, pessaries, pharmacotherapy, sacral colpopexy, sacrospinous ligament suspension, uterosacral ligament suspension, illiococcygeal suspension, anterior colporrhaphy, posterior colporrhaphy, enterocele repair, perineorrhaphy, Burch procedure, total hysterectomy, supracervical hysterectomy, and mid-urethral sling procedure.

8. The method as in claim 1, wherein said steps (d) and (e) further comprising returning and visualizing said medical diagnosis, the probabilities of success for said possible treatments and the adapted three-dimensional model to a medical device which was used in acquisition of said health data for the female pelvic floor.

9. The method as in claim 1, wherein said step (f) further comprising making a decision about pelvic floor treatment of the patient based on maximum probability of success for at least one of said possible treatments.

10. The method as in claim 1, wherein said step (g) further comprising a set of said dated pages in said digital twin mapping of physical and virtual data during a life cycle of the female pelvic floor.
